(19) United States
(12) Patent Application Publication
(10) Pub. No.: US 2005/0084889 A1
(43) Pub. Date: Apr. 21, 2005

(54) Preferential display

(75) Inventors: Robert C. Chin, Austin, TX (US); Gregory F. Lopreato, Austin, TX (US)

Correspondence Address: TOLER & LARSON & ABEL L.L.P., 5000 PLAZA ON THE LAKE STE 265, AUSTIN, TX, 78746, US

(73) Assignee: GeneXpress Informatics, Inc.

(21) Appl. No.: 10/959,749

(22) Filed: Oct. 6, 2004

Related U.S. Application Data
(63) Continuation-in-part of application No. 09/961,089, filed on Sep. 24, 2001, now Pat. No. 6,861,219.
(60) Provisional application No. 60/234,751, filed on Sep. 25, 2000.

(51) Int. Cl. C12Q 1/68

(52) U.S. Cl. 435/6

(57) ABSTRACT

The disclosure is directed to a method for comparing a first tissue sample and a second tissue sample. The method includes degrading hybrid duplexes from a solution to leave at least one of a first set of uncomplexed RNA strands derived from the first tissue sample and a first set of uncomplexed cDNA stands derived from the second tissue sample. The solution includes the hybrid duplexes, the first set of uncomplexed RNA strands and the first set of uncomplexed cDNA strands.

### CROSS-REFERENCE TO RELATED APPLICATION(S)

This application is a Continuation-in-Part Application of U.S. patent application Ser. No. 09/961,089 entitled “Preferential Display” naming inventors Shahzi Iqbal and Robert Chin, which claims priority of U.S. Patent Application Ser. No. 60/234,751, filed Sep. 25, 2000 entitled: “Preferential Display”, both of which are incorporated herein by reference in their entirety.

### FIELD OF THE DISCLOSURE

This invention, in general, relates to analysis of gene expression. More specifically, this invention relates to the preferential display of differences between two gene expression samples.

### BACKGROUND

Methods for assaying gene expression can be classified into two major types: open methods, which do not require prior knowledge of the genes being measured, and closed methods, which measure expression levels of already collected clones or sequences. Some expression analysis techniques only measure on a gene-by-gene basis while others assay multiple genes simultaneously. Finally, some methods directly measure differential expression between two samples and some examine expression levels from one sample at a time, followed by computation-based comparisons. Regardless of the methods chosen researchers identify or access through databases vast quantities of expression information to find the actual cause and effect on the gene expression.

The history of gene expression analysis began when laboratory methods were developed to examine expression of individually known genes. The northern blot technique, introduced in 1977, hybridizes labeled DNA or RNA of known genes to RNA blots. The resulting expression patterns of mRNA transcripts were then read. This technique is still widely used to confirm the results of other types of gene expression studies. In 1977, another method was published that protects a DNA-labeled probe against degradation by the single-stranded nuclease S1 if the probe is annealed to an RNA. Later, RNase protection assays were developed to detect the expression of specific, previously characterized RNA and to compare their levels of expression. With this technique, a specific labeled antisense RNA forms a hybrid with its corresponding mRNA. When exposed to a single-strand-specific nuclease, the hybrids resist degradation and can be detected using gel electrophoresis. A later approach, differential plaque-filter hybridization, can detect differences in the expression of cloned cDNA between two samples.

In 1993, subtractive hybridization techniques became available for constructing subtractive cDNA libraries. This methodology hybridizes cDNA from one pool to mRNA from another. Then, cDNA libraries are constructed from the transcripts that are not hybridized, these being used to identify specific MRNA. A modification of this technique, representational difference analysis (RDA), also uses preferential amplification of non-subtracted fragments. In RDA, “representations” or simplified versions of the genomes being studied (amplicons) are created using restriction digestion. This method was first developed to examine the differences between genomes, but has proven useful for cloning differentially expressed genes. From this method, suppressive subtractive hybridization (SSH) was derived, which enables further suppression amplification of non-subtracted fragments. SSH combines normalization (equalizing the abundance of cDNAs within the target population) and subtraction (excluding the common sequences between the target and driver populations) in a single procedure. Results from both RDA and SSH should be validated using other methods.

Early gene expression methods, such as those already mentioned, are relatively small-scale techniques. They either focus on measuring mRNA expression levels for individual, well-characterized genes, or use in vitro nuclear “run-on” transcription assays to determine the transcriptional profiles of several active genes simultaneously. They are therefore inadequate for conducting large-scale screening and developing expression profile patterns for tissues or cells (the basic requirements for efficient pharmaceutical research). Thus, several newer methods for high-throughput screening (HTS) have been developed over the past decade, including differential display, expressed sequence tag (EST) methodology and many array techniques. Collectively, they have made it possible to identify the expression levels of novel genes and characterize them, correlate mRNA expression patterns in many tissue types with disease states, identify side effects of current and experimental treatments, and determine the effects of compounds on non-target tissues.

Differential display of eukaryotic mRNA, first reported in 1992, was a major advance in the comparison of gene expression differences between cells or tissues. Encompassing the use of either arbitrarily or specifically primed PCR, it is perhaps the most widely used method involving gel electrophoresis for comparing gene expression. Both methods amplify partial cDNAs from subsets of mRNA samples by using reverse transcription and PCR. These short cDNA fragments are then typically displayed on polyacrylamide gels. Differential display can simultaneously measure both up- and down-regulation across tens of samples.

Originally, this method used an oligo(dT) primer with an anchor of one or two bases at the 3′ terminal. Reverse transcription and denaturation were followed by arbitrary priming on the resulting first strand of cDNA. A series of products were then derived from the 3′ end of the MRNA by using PCR with the original primer (a radiolabeled nucleotide) and a set of short, random decamer primers. Each random primer annealed to the mRNA at a different position relative to the anchor primer. Products showing significant differential expression were sequenced, after size fractionation of the PCR sample using denaturing gel electrophoresis, generally after overnight autoradiographic exposure.

DNA microarrays measure expression by using templates containing hundreds or thousands of probes that are exposed simultaneously to a target sample. They make it possible to systematically survey DNA and RNA variation for the first time and are becoming a standard tool for drug discovery and evaluation. Microarray techniques are so powerful that their uses are often limited largely by the challenge of managing and analyzing the data they generate. DNA microarray technology evolved from a paper published in 1975 by E.M. Southern (the originator of the Southern blot), who showed how a solid support could be used to examine nucleic acids. This was advanced by the development of non-porous solid supports, leading to miniaturization and the use of fluorescence-based detection methods. The two main types of templates are long DNA fragments (over 100 base pairs) and oligo-nucleotides (generally 18-25 mers). Microarrays are expensive, although efficiencies should improve and costs should drop dramatically in the next couple of years, enabling these tools to become accessible to most research laboratories. Besides cost, microarrays are limited by the fact that they can only probe genes for which clones or sequences are already available. Further-more, their accuracy can be limited by the purity of the RNA and the quantity of RNA for each hybridization.

By understanding gene expression patterns, researchers can gain information that can link sites of expression, bio-chemical pathways, and normal or pathological functions in organs and whole organisms. Because of their speed and breadth, microarrays should impact genetic profiling in several ways: Accelerate the understanding of the molecular basis of disease or environmental stresses, Improve knowledge of model systems, Explore pathogens, pathogenic, environmental (microgravity) reactions in terms of gene expression, Pinpoint new molecular level explanations to environmental effects, and Examine efficacy and toxicity responses to environmental or other external simulates.

Microarrays have already determined how several important genes are abnormally regulated in disease. For example, a microarray of approximately 100 genes that have a role in inflammation was used to examine rheumatoid tissue. This revealed upregulation of the genes encoding interleukin-6 and several matrix metalloproteinases. In another instance, a novel gene involved in promoting tumors was discovered by using a 1000-element micro-array of unknown cDNAs to examine how treatment with phorbol esters affects expression levels. Microarrays should provide more detailed knowledge about pathogens by systematically examining every gene in a microbe to uncover the overall expression pattern. In addition, microarrays will continue to contribute to the understanding of responses to drug treatments. For example, a recent study used microarrays to measure the effects of kinase inhibitors on the entire yeast genome by measuring changes in mRNA levels before and after treatment. In another example, microarray studies of yeast cells showed that the immunosuppressive drug FK506 had the same effect on gene expression level patterns as ablation of the gene that FK506 suppresses. Furthermore, this study showed that, in the absence of this gene, FK506 affected expression levels in other ways. This suggests that the drug might have more than one target. Microarrays are also proving useful in the determination of drug toxicity.

Expression profiling using cDNA microarrays begins by arraying many gene-specific amplicons derived from the cDNA clones onto a single matrix. Using two-color hybridization, cDNA representations of total RNA pools are created from test and reference cells, fluorescently tagged with two different colors, then mixed together before being hybridized to the matrix. For each transcript, the resulting fluorescence signals reflect the difference in abundance between the two samples. Two-color hybridizations provide rapid comparisons between the two samples, but they do not measure the absolute levels of gene expression for either sample. By contrast, one-color hybridization is slightly slower, as hybridizations of the two samples must be performed separately to reach meaningful comparisons. However, each one-color hybridization measures absolute levels of gene expression rather than comparative levels. After these actual levels are recorded in databases, they can be compared with levels from other samples without the need to perform comparative experiments. Although performing 1000 two-color hybridizations results in 1000 pair-wise comparisons, conducting 1000 one-color hybridizations yields almost half-a-million pair-wise comparisons, as the absolute values of one-color hybridizations can be evaluated against each other.

Using either the one- or two-color methods, microarray experiments must be performed repeatedly to ensure accuracy of the data. However, computational averaging of the signals of one-color hybridizations from multiple independent samples is more straightforward. The choice between using one-color versus two-color methods depends on several factors, including the number of transcripts under examination, the need for speedy results and cost differences. Hence, one-color hybridizations are often more useful for surveying a large number of genes, while two-color hybridizations can be preferable for more restricted sampling.

### BRIEF DESCRIPTION OF THE DRAWINGS

The present disclosure may be better understood, and its numerous features and advantages made apparent to those skilled in the art by referencing the accompanying drawings.

FIGS. 1, 2, and 3 are flow diagrams illustrating an exemplary preferential display method.

The use of the same reference symbols in different drawings indicates similar or identical items.

### DESCRIPTION OF THE PREFERRED EMBODIMENT(S)

The present method is a gene display technology that provides a simplified, cost-effective method to efficiently identify and accurately isolate—differentially expressed gene sequences between normal and diseased states of tissues. The method utilizes a combination of biomolecular chemistry methods to eliminate redundant sequences and radiolabeled assay to identify unique sequences found in normal and/or diseased tissues. The method can be implemented in a single reaction tube, is amendable to miniaturization, and is extremely cost-effective which would be of benefit in toxicogenomic and drug discovery applications. It is estimated redundant sequences amount to 98% of the total of all genes expressed in current display technologies.

The disclosure is directed to a preferential display method. The method may include sample collection and categorizing of the cells, tissues or blood samples. For example, two samples are collected including a sample of normalized control, cells and a sample of disease state cells. Once the normalized control cells are prepared in isolation from the diseased state cells, expressed RNA, such as mRNA, is isolated for each sample. In one exemplary embodiment, the method uses Total RNA isolation and purification from cells/tissue using Totally RNA (RNA extraction kit from Ambion (Austin, Tex.)). Reactions are carried out per manufacturer's instructions. A portion of at least one of the RNA samples is reverse transcribed to produce cDNA. RNase is then added to digest the single stranded RNA's in each tube. The Rnase is then inactivated using Proteinase K. The Proteinase K is then removed by phenol/chloroform extraction. At this point, RNA samples of normal and/or RNA samples of diseased tissue are added to their complementary tubes, normal cDNA with diseased RNA and/or diseased cDNA with normal RNA. Common sequences in each tube hybridize to form cDNA/RNA complements. The tubes are then treated with an enzyme or combination of enzymes, such as enzymes selected from the group consisting of Exonuclease III, Exonuclease VII, T7 Exonuclease, and S1 Nuclease, to degrade the hybridized complements. The remaining undigested cDNA are unique sequences expressed in either normal or diseased states. Differential display techniques, gene display techniques, and PCR may then be performed on the remaining mixture of cDNA.

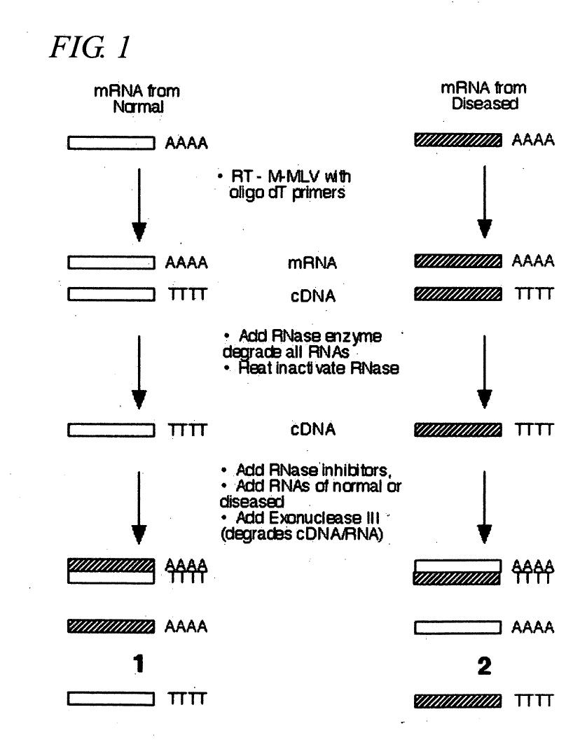

FIG. 1 illustrates two possible paths for performing the method. A sample solution including mRNA is reverse transcribed. In one exemplary embodiment, an mRNA sample is derived from normal or control cells. In an alternative example, an mRNA sample is derived from diseased tissue.

In one exemplary embodiment, the mRNA sample is reverse transcribed using reverse transcriptase (RT). The reverse transcription process may use dye labeled anchored oligio dT primers, such as florescent dye labeled oligio dT primers, or may use radioactive isotope labeled nucleic acids. Alternatively, the reverse transcription process is performed free of labeling. As a result of the reverse transcription, the sample solution includes cDNA strands associated with or derived from the same tissue. In one particular embodiment, the cDNA strands are labeled.

The mRNA within the sample solution may be degraded. For example, RNase may be added to the sample solution to degrade the mRNA. In one exemplary embodiment, the RNase is subsequently deactivated through the addition of proteinase K or through heat inactivation. A phenol chloroform extraction may be performed to remove Proteinase K. In another exemplary embodiment, RNase inhibitor is added to the solution. As a result of the mRNA degradation, the sample solution includes uncomplexed single strand cDNA strands.

Another solution including uncomplexed mRNA is derived from a second tissue sample. For example, if the first tissue sample is normal or control tissue, the second tissue may be a diseased tissue. Alternatively, if the first tissue sample is a diseased tissue sample, the second tissue sample may be a normal or control tissue sample. The second solution including uncomplexed mRNA derived from the second tissue sample is added to the first sample solution. Optionally, RNase inhibitor may be added to the second solution before addition to the first sample solution or in conjunction with the addition of the second solution.

As a result of the addition, subsets of the mRNA strands having complementary sequences with subsets of the cDNA strands will complex to form hybrid duplexes. Other subsets of the mRNA and cDNA will remain as uncomplexed single strands, such as those subsets that include non-complementary sequences.

An enzyme may be added to degrade the hybrid duplexes. In one exemplary embodiment, the hybrid duplexes are degraded, leaving uncomplexed non-complementary sequences of cDNA and MRNA. In other exemplary embodiment, the hybrid duplexes and one of the single stranded cDNA or the single stranded mRNA are degraded, leaving one of the single stranded cDNA or single stranded mRNA. The duplex may be degraded using an enzyme or combination of enzymes selected from the group consisting of Exonuclease III, Exonuclease VII, T7 Exonuclease, and S1 Nuclease.

In one particular embodiment, the sample solution includes a subset of the single stranded cDNA, such a dye labeled cDNA, absent those sequences of cDNA having complements in the second tissue sample mRNA sequences. In an alternative example, the uncomplexed mRNA may remain.

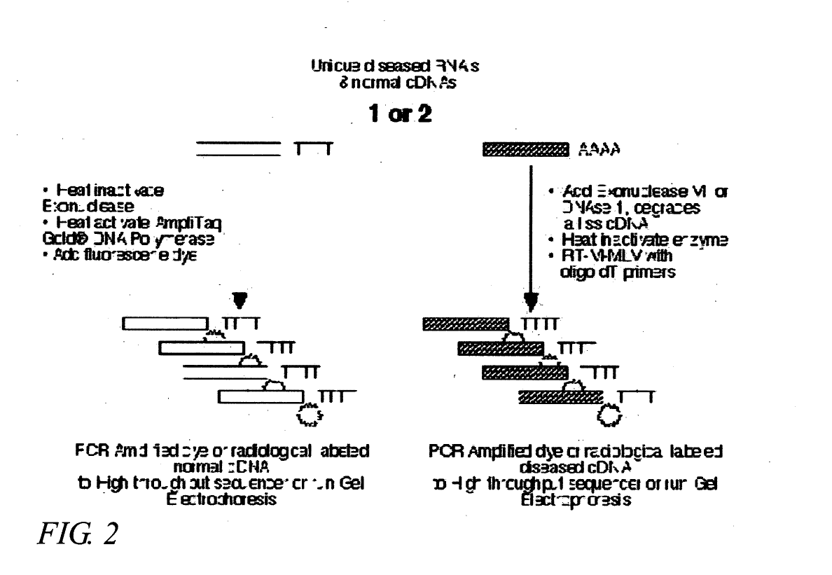

Using the solution including the subset of single stranded cDNA, as shown in FIG. 2, the subset of cDNA may be amplified using PCR. Alternatively, RNA may be reverse transcribed. Differential display techniques may also be used to duplicate a reduced sampling of genes that are characteristic of the differences between the tissue samples. Dye labeled primers or radioactive isotope labeled nucleic acids may be used in the PCR process or differential display techniques.

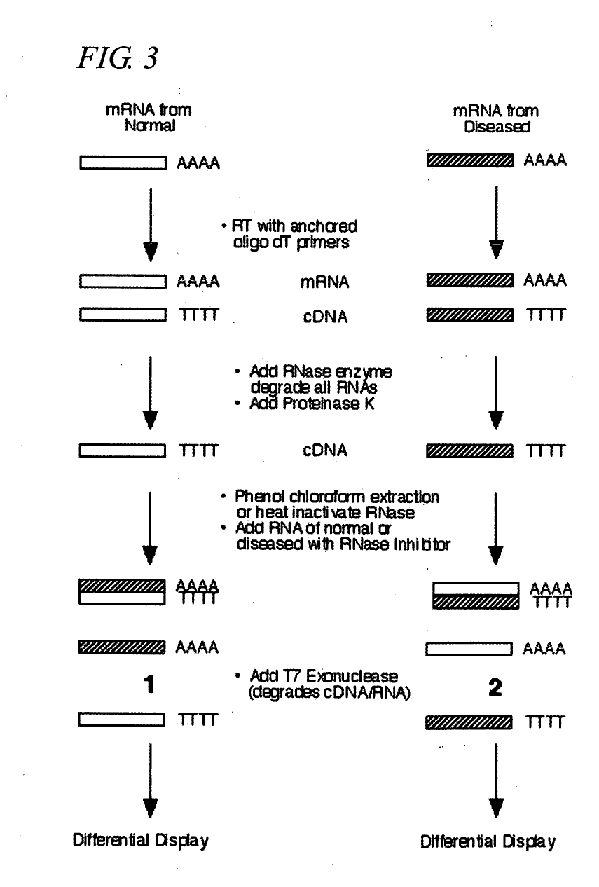

FIG. 3 illustrates another exemplary embodiment of a method. The method may follow one or both paths illustrated. For example, the method may use a first tissue sample, such as normal or control tissue, and a second tissue sample, such as diseased tissue. In this exemplary embodiment, a sample solution that includes mRNA is derived from tissue. The mRNA is reverse transcribed using reverse transcriptase (RT). RNase is added to degrade the mRNA and proteinase K is added to degrade or deactivate the RNase. A phenol cholorform extraction is used to remove the Proteinase K or heat inactivation of the RNase may be used. RNase inhibitor and RNA derived from a second tissue is added to the sample solution.

Sequences of RNA combine with complementary cDNA sequences to form hybrid duplexes. Sequences of RNA and cDNA that are not complementary do not form complexes. In one exemplary embodiment, T7 Exonuclease is used to degrade the hybrid duplexes to leave at least one of the uncomplexed RNA and the cDNA without complementary sequences of RNA. The cDNA may then be used in a differential display technique. In one particular embodiment, the cDNA is used in a differential display technique in which fluorescent dye labeling or radioactive isotope labeling is used.

In one exemplary embodiment, the disclosure is directed to a method for comparing a first tissue sample and a second tissue sample. The method includes degrading hybrid duplexes from a solution to leave at least one of a first set of uncomplexed RNA strands derived from the first tissue sample and a first set of uncomplexed cDNA stands derived from the second tissue sample. The solution includes the hybrid duplexes, the first set of uncomplexed RNA strands and the first set of uncomplexed cDNA strands.

In another exemplary embodiment, the disclosure is directed to a method to eliminate complementary cDNA and RNA sequences found in two different tissue types. The method includes degrading cDNA/RNA hybrid duplexes using S1 nuclease enzyme.

In a further exemplary embodiment, the disclosure is directed to a method to eliminate complementary cDNA and RNA sequences found in two different tissue types. The method includes degrading cDNA/RNA hybrid duplexes using Exonuclease enzyme.

In a particular embodiment, the disclosure is directed to a kit including a first reagent solution including reverse transcriptase, a second reagent solution including RNase, and a third reagent solution. The third reagent solution is configured to degrade hybrid duplexes when mixed with a sample solution to leave at least one of a first set of uncomplexed RNA strands derived from a first tissue sample and a first set of uncomplexed cDNA strands derived from a second tissue sample. The sample solution includes the hybrid duplexes, the first set of uncomplexed RNA strands and the first set of uncomplexed cDNA strands.

The sample solution may be formed by mixing a first tissue sample solution and a second tissue sample solution. The first tissue sample solution includes the first set of uncomplexed RNA strands and a second set of RNA strands. The second tissue sample solution includes the first set of uncomplexed cDNA strands and a second set of cDNA strands. The second set of RNA strands form the hybrid duplexes with the second set of cDNA strands. In one particular embodiment, the third reagent solution includes an enzyme selected from a group consisting of Exonuclease III, Exonuclease VII, T7 Exonuclease, and S1 Nuclease. The kit may also include a reagent solution including proteinase K and/or a reagent solution including RNase inhibitor. The kit may also include instructions for performing a method comprising degrading hybrid duplexes when mixed with the sample solution to leave the at least one of the first set of uncomplexed RNA strands and the first set of uncomplexed cDNA strands.

### EXAMPLE

In one exemplary embodiment, the method is performed as described below. The materials used in this exemplary embodiment include:

Materials:

dH₂O
10X PCR Buffer (100 mM Tris-Cl, pH 8.4, 500 mM KCl, 15 mM,
15 mM MgCl₂ and 0.01% gelatin),
GeneHunter Corporation ™, Nashville, TN
5X RT Buffer (125 mM Tris-Cl, pH 8.3, 188 mM KCl, 7.5 mM MgCl₂
and 25 m m DTT), GeneHunter Corporation ™, Nashville, TN
dNTP (250 μM) and (25 μM) GeneHunter Corporation ™, Nashville,
TN
H-T₁₁M, where M could be G, A or C (2 μM), GeneHunter
Corporation ™, Nashville, TN
H-APX, where X could be any arbitrary primer from 1 to 80 (2 μM),
GeneHunter Corporation ™, Nashville, TN
MMLV Reverse Transcriptase (100 U/μl), GeneHunter Corporation ™
RNAse H (60 U/μl), diluted to 4 U/μl with TE buffer, Takara, Shiga,
Japan
Taq DNA Polymerase (5 U/μl) Qiagen ™, Valencia, CA
Taq Polymerase Master Mix, Qiagen ™, Valencia, CA
Exonuclease III (65 U/μl), New England Biolabs ™ Life
Technologies ™, Carlsbad, CA
T7 Exonuclease (152 U/μl), New England Biolabs ™ Life
Technologies ™, Carlsbad, CA
P₃₃α ATP (250 μCi), NEN ® Life Science Products, Boston, MA
cDNA (0.01 μg/μl)
RNA (0.1 μg/μl)

The Reverse Transcription protocol was obtained from the RNAimage kit manufactured by the GenHunter Corporation™, Nashville, Tenn. The solution included the following components.

| Component | Amount |
|---|---|
| dH₂O | 9.4 μl |
| 5X RT Buffer | 4.0 μl |
| dNTP (250 μM) | 1.6 μl |
| H-T₁₁M (2 μM) | 2.0 μl |
| Total RNA (0.1 μg/μl) | 2.0 μl |
| MMLV RT | 1.0 μl |

The reverse transcription protocol included the following process.

Thermocycling Conditions for the RT Reaction

65° C. for 5 minutes
37° C. for 10 minutes
*Pause: Add MMLV RT Enzyme
37° C. for 50 minutes
75° C. for 5 minutes
Hold at 4° C.

Following the RT reaction from Example 1, an RNAse H incubation is performed by adding 1.0 μl of RNAse H (4 U/μl) to the 20 μl RT Reaction product:

| Component | Amount |
|---|---|
| 10 X RNAse H buffer | 2.0 μl |
| cDNA/mRNA complex (0.01 μg/μl) | 17.0 μl |
| RNAse H | 1.0 μl |

The RNAse is then heat inactivated at 94 C for 5 minutes. Alternatively, the Rnase is degraded with Proteinase K. The Proteinase K is then phenol chloroform extracted out of the sample. RNase inhibitor is then added.

At this point the cDNA is ready to be mixed with the foreign RNA. For example, if brain cDNA is obtained, then heart RNA can be introduced and vice versa. The brain cDNA is allowed to hybridize with the heart RNA and vice versa.

Two different embodiments are described below depending on the enzyme utilized. The first embodiment is the Exonuclease III embodiment (digests dsDNA and portions of the DNA/RNA complex) and the other embodiment is the T7 Exonuclease (digests dsDNA and the DNA/RNA complex).

Exonuclease III Approach

The solution below is added to the sample solution including the hybrid duplexes and single stranded mRNA and cDNA.

| Component | Amount |
|---|---|
| EXO III Buffer | 2.0 μl |
| cDNA/RNA complex + sscDNA | 16 μl |
| Exonuclease III | 2.0 μl |

The process includes:

Thermocycling Conditions for the Exonuclease III Reaction

95° C. for 10 seconds
*Pause:
60° C. for 3 minutes
37° C. for 10 seconds
*Pause: Add Exonuclease III
37° C. for 30 minutes
95° C. for 10 minutes
Hold at 4.0° C.

T7 Exonuclease Approach

The solution below is added to the sample solution including the hybrid duplexes and single stranded MRNA and cDNA.

| Component | Amount | |
|---|---|---|
| RNAse Inhibitor | 1.0 | μl |
| T7 Exonuclease Buffer | 2.0 | μl |
| cDNA/RNA complex | 14.0 | μl |
| T7 Exonuclease | 3.0 | μl |
| Exonuclease III | 2.0-3.0 | μl (Optional) |
| S1 Nuclease | 2.0-10.0 | μl (Optional) |

The above-disclosed subject matter is to be considered illustrative, and not restrictive, and the appended claims are intended to cover all such modifications, enhancements, and other embodiments, which fall within the true scope of the present invention. Thus, to the maximum extent allowed by law, the scope of the present invention is to be determined by the broadest permissible interpretation of the following claims and their equivalents, and shall not be restricted or limited by the foregoing detailed description.

1. A method for comparing a first tissue sample and a second tissue sample, the method comprising:
degrading hybrid duplexes from a solution to leave at least one of a first set of uncomplexed RNA strands derived from the first tissue sample and a first set of uncomplexed cDNA stands derived from the second tissue sample, the solution including the hybrid duplexes, the first set of uncomplexed RNA strands and the first set of uncomplexed cDNA strands.

2. The method of claim 1, further comprising:
forming the solution by mixing a first tissue sample solution and a second tissue sample solution, the first tissue sample solution including the first set of uncomplexed RNA strands and a second set of RNA strands, the second tissue sample solution including the first set of uncomplexed cDNA strands and a second set of cDNA strands, the second set of RNA strands forming the hybrid duplexes with the second set of cDNA strands.

3. The method of claim 2, further comprising:
deriving the first tissue sample solution from the first tissue sample.

4. The method of claim 3, further comprising adding RNase inhibitor to the first tissue sample solution.

5. The method of claim 2, further comprising:
deriving uncomplexed RNA strands from the second tissue sample.

6. The method of claim 5, further comprising:
reverse transcribing the uncomplexed RNA strands derived from the second tissue sample to form the second tissue sample solution.

7. The method of claim 6, further comprising:
within the second tissue sample solution, degrading the uncomplexed RNA strands derived from the second tissue sample using RNase.

8. The method of claim 7, further comprising:
adding proteinase K to the second tissue sample solution after degrading the uncomplexed RNA strands derived from the second tissue sample.

9. The method of claim 1, further comprising:
deriving labeled cDNA from the at least one of the first set of uncomplexed RNA strands and the first set of uncomplexed cDNA strands.

10. The method of claim 1, wherein one of the first tissue sample and the second tissue sample is a normal tissue sample.

11. The method of claim 10, wherein one of the first tissue sample and the second tissue sample is a diseased tissue sample.

12. The method of claim 1, wherein degrading the hybrid duplexes is performed using an enzyme selected from a group consisting of Exonuclease III, Exonuclease VII, T7 Exonuclease, and S1 Nuclease.

13. A kit comprising:
a first reagent solution comprising reverse transcriptase;
a second reagent solution comprising RNase; and
a third reagent solution configured to degrade hybrid duplexes when mixed with a sample solution to leave at least one of a first set of uncomplexed RNA strands derived from a first tissue sample and a first set of uncomplexed cDNA strands derived from a second tissue sample, the sample solution including the hybrid duplexes, the first set of uncomplexed RNA strands and the first set of uncomplexed cDNA strands.

14. The kit of claim 13, wherein the sample solution is formed by mixing a first tissue sample solution and a second tissue sample solution, the first tissue sample solution including the first set of uncomplexed RNA strands and a second set of RNA strands, the second tissue sample solution including the first set of uncomplexed cDNA strands and a second set of cDNA strands, the second set of RNA strands forming the hybrid duplexes with the second set of cDNA strands.

15. The kit of claim 13, wherein the third reagent solution includes an enzyme selected from a group consisting of Exonuclease III, Exonuclease VII, T7 Exonuclease, and S1 Nuclease.

16. The kit of claim 13, further comprising a fourth reagent solution including proteinase K.

17. The kit of claim 13, further comprising a fourth reagent solution including RNase inhibitor.

18. The kit of claim 13, further comprising instructions for performing a method comprising degrading hybrid duplexes when mixed with the sample solution to leave the at least one of the first set of uncomplexed RNA strands and the first set of uncomplexed cDNA strands.

19. A method to eliminate complementary cDNA and RNA sequences found in two different tissue types, the method comprising:
degrading cDNA/RNA hybrid duplexes using S1 nuclease enzyme.

20. A method to eliminate complementary cDNA and RNA sequences found in two different tissue types, the method comprising:
degrading cDNA/RNA hybrid duplexes using Exonuclease enzyme.
